(19) United States
(12) Patent Application Publication
(10) Pub. No.: US 2009/0216049 A1
(43) Pub. Date: Aug. 27, 2009

(54) Process and intermediates for the production of BDNPA and BDNPF and other bis(dinitroalkyl)acetals and formals

(75) Inventors: Renato R. Rindone, Fair Oaks, CA (US); Michael Kenneth Free, Placerville, CA (US); Robert Allan Morrison, JR., Sacramento, CA (US)

Correspondence Address: Renato Rindone;Dimension Technology Chemical Systems Inc., PO Box 2557, Fair Oaks,, CA, 95628, US

(73) Assignee: Dimension Technology Chemical Systems, Inc., Fair Oaks, CA (US)

(21) Appl. No.: 11/409,330

(22) Filed: Apr. 20, 2006

(51) Int. Cl. C07C 205/02

(52) U.S. Cl. 568/590

(57) ABSTRACT

Bis(dinitroalkyl)acetals and formals having the formula (1)

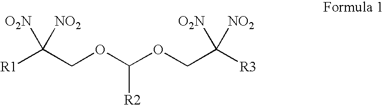

particularly bis(2,2-dinitropropyl)acetal and bis(2,2-dinitropropyl)formal are produced by oxidative nitration of compounds having the formula (2):

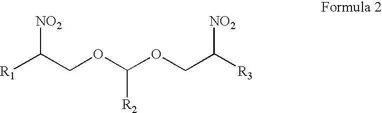

preferably via a sodium or other alkali metal, or alkaline earth metal, salt of compounds of Formula (2). Certain of the compounds of formula (1) are novel. All of the intermediates of formula (2) are novel and form another aspect of this invention, as does a process for their production by reacting an aldehyde with a nitroalkanol. The process can readily produce a mixture of the dinitro compounds known as BDNPA and BDNPF (also known as A/F) using the non-explosive intermediates, BNPA and BNPF, both of which are novel.

### BACKGROUND OF THE INVENTION

This invention relates to the preparation of geminal-dinitro compounds, namely bis-dinitroalkyl acetals and formals, as described herein, which are used in rocket propellants and explosives, among other purposes. This invention includes novel intermediates for such compounds, as well as novel processes for preparing the compounds from the intermediates, and for preparing the intermediates. This invention also includes some novel geminal dinitro compounds.

In about January of 1946, the Department of the Navy, Office of Naval Research funded a significant effort to develop a smokeless propellant based on polynitro-containing organic compounds at GenCorp Aerojet. Documentation of this Navy-funded effort, extending into about 1963 at GenCorp-Aerojet, can be found in a number of declassified Navy reports. Although this work was originally classified as confidential, it was officially declassified by order of the Department of the Navy, Office of Naval Research (ONR:426:RLH:10, April, 1961 and EO11652, December, 1972) and was later published. This research program resulted in the discovery of new synthetic routes and manufacturing methods for the preparation of geminal dinitro compounds. Most notably, a high-energy, two-component nitroplasticizer, composed of an approximately 50/50 weight % eutectic solution of bis(2,2-dinitropropyl)acetal [BDNPA] and bis(2,2-dinitropropyl)formal [BDNPF], referred to hereinafter as A/F, was prepared and successfully used in the Polaris missile system. —The A/F composition can contain from about 45 to about 55 weight % of one component and from about 55 to about 45 weight % of the other, as reflected in the current military specification (Weapon Specification 1141A and subsequent amendments).

More recently, the U.S. Army Tank-automotive and Armaments Command—Armament Research Development and Engineering Center (TACOM-ARDEC) Explosives Research and Technology Team has found it advantageous to use the energetic plasticizer A/F in explosive formulations. This use of A/F allows for the development of explosive compositions that are less sensitive to initiation by outside stimuli while maintaining operational performance.

In addition to the work conducted at Aerojet, other investigators at the Office of Naval Research, Purdue University and Ohio State University also gave major contributions leading to the development of energetic nitro compounds. Since about 1962, the work done by V. Grakauskas and K. Baum at Fluorochem, M. Kamlet at NWC-Silver Springs, H. Adolph at NSWC-Indian Head, M. Frankel at Rocketdyne, H. Feuer and N. Komblum at Purdue University, G. Olah at The University of Southern California, A. Nielsen at NWC-China Lake and J. Boyer at the University of New Orleans has greatly expanded the technology to prepare aliphatic and heterocyclic nitro and polynitro compounds. For a deeper understanding of this chemistry, see articles, reviews and texts on the chemistry of nitro compounds such as N. Ono, The Nitro Group in Organic Synthesis (and references therein), Wiley-VCH (2001); Noble, F. G. Jr., et. al., Chem. Reviews, 64(1):19-58 (1964); Feuer, H. et al., The Chemistry of the Nitro and Nitroso Groups, Part 1-; Part 2-, Interscience Publishers 1969; (1970); Patai, S. Supplement F, The Chemistry of Amino, Nitroso, and Nitro Compounds and their Derivatives, Parts 1 and 2, John Wiley & Sons (1982); Torsell, K. B. G. Nitrile Oxides, Nitrones, and Nitronates in Organic Synthesis, VCH (1988); Breuer, E. H. G. Nitrones, Nitronates and Nitroxides, John Wiley & Sons (1989); Barrett A. G. M. Tetrahedron, “Nitroalkanes and Nitroalkenes in Synthesis”, 46(21) (1990); and Olah, G. et. al., Nitration, Methods and Mechanisms, VCH (1994).

In the early Navy program effort, the outcome was the development of two different chemical processes to prepare A/F, one by Gencorp-Aerojet (Sacramento) and the other by the Navy (Indian Head). Both processes used nitroethane as the starting material and both processes prepared 2,2-dinitropropanol (DNPOH) as the key intermediate. The Aeroj et process [Hamel, E. E. et. al., Ind. Eng. Chem. Prod. Res. Devel., 1: 108-116 (1962)] used an improved ter Meer [ter Meer, E. Ann., 181:1 (1876)] reaction to prepare the DNPOH. The Navy process used oxidative nitration technology developed by Kaplan and Shechter [Kaplan, H. Shechter, J. Am. Chem. Soc., 83:3535 R. B. (1961)] to prepare DNPOH. The Aerojet process was scaled-up to continuous-flow production plant equipment in 1962 and the Navy process was scaled-up to batch size production plant equipment a little earlier. Continued studies [Komblum, et. al., J. Org. Chem., 48:332 (1983)] showed that geminal-dinitro compounds could be formed via a radical-anion mechanism by reacting a nitro-olefin with nitrite ion using stoichiometric quantities of potassium ferricyanide. Grakauskas and co-workers improved the economics of this process [Garver, L. et al., J. Or Chem., 50, 1699, U.S. Pat. Nos. 4,594,430; 4,774,366; 4,910,322 (1985; 1986; 1988; 1990)] by using catalytic quantities of potassium ferricyanide and a stoichiometric amount of oxidant, such as sodium persulfate, to keep iron (Fe) present in the trivalent state. Using the technology developed by Grakauskas and Baum, Alliant Techsystems, Thiokol Corporation built a production plant to produce A/F. Thiokol also obtained U.S. Pat. Nos. 5,449,835 and 5,648,556 on producing BDNPF and BDNPA by reacting DNPOH with formaldehyde and acetaldehyde, respectively, with an acid catalyst in the presence of a non-chlorinated solvent.

A reaction scheme for the ter Meer process is shown in Scheme I below. A reaction scheme for the oxidative nitration process is shown in Scheme II. A reaction scheme for the ferricyanide process is shown in Scheme III.

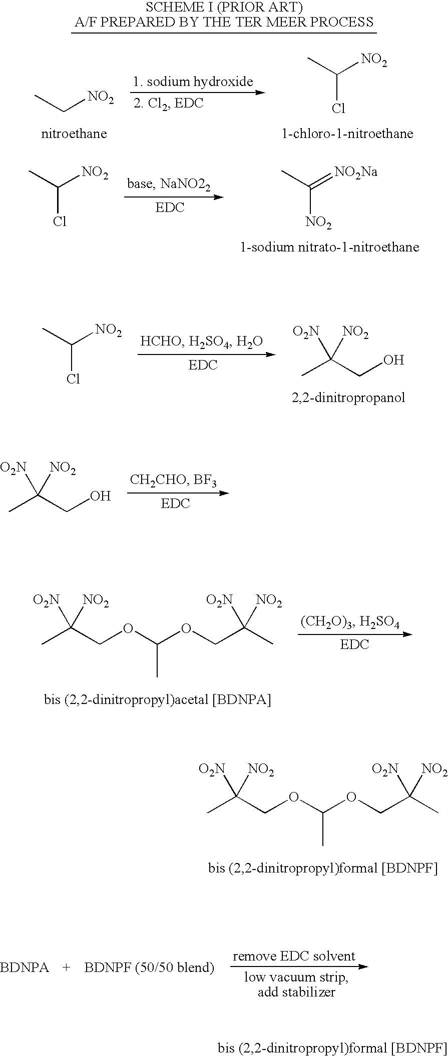

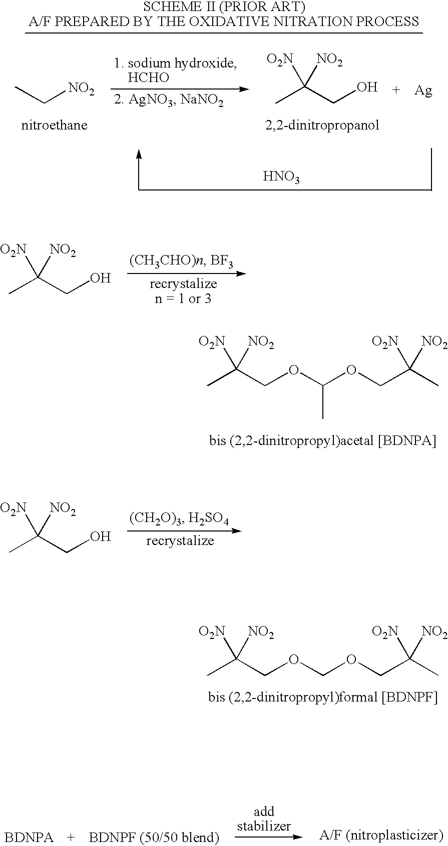

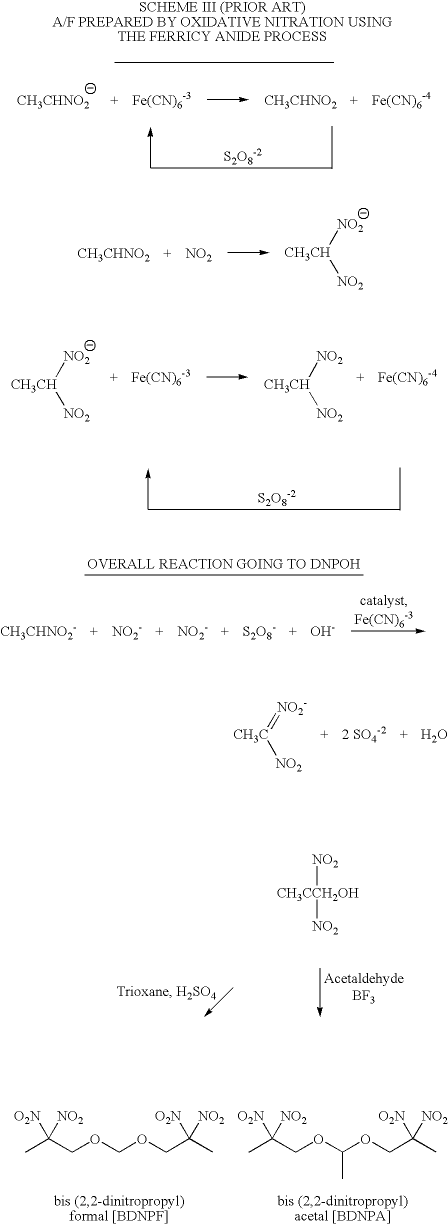

There are advantages and disadvantages for all of the above-mentioned processes. The Aerojet continuous process was designed to manufacture about 1 million pounds/year of A/F using six processing steps. The overall A/F yield of this process was about 45% to 50% and the number of process impurities was considerably higher than the impurities formed in the four-step oxidative nitration process shown in Scheme 2. The oxidative nitration process had a higher overall yield, of about 60%, but because the process used silver nitrate and because the individual components, BDNPA and BDNPF, were recrystallized before blending, it had a significantly higher operating cost and the A/F productivity was only a fraction of the Aerojet continuous process. The oxidative nitration process using ferricyanide also produced A/F that was high in low-boiling impurities, one of which [2,6-dimethyl-4-(2,2-dinitropropoxyl)-1,3-dioxane] has been found to be the major impurity responsible for poor A/F shelf life and aging performance of the end-use product containing the A/F. All three of these processes prepare A/F by way of the intermediate 2,2-dinitropropanol (DNPOH), a class-1.1 explosive that is subject to explosive-quantity-distance restrictions during the construction of the plant.

### BRIEF SUMMARY OF THE INVENTION

This invention overcomes major problems inherent with the processes previously known or used to make A/F, and provides novel processes and intermediates. In addition, the novel processes and intermediates are suitable for producing the individual compounds comprising A/F and compounds related to A/F. The starting materials are either commercially available or can readily be synthesized. In addition, none of the intermediates prepared and described in this invention are explosive as defined by the Department of Defense TB 700-2. [Department of Defense Ammunition and Explosives Hazard Classification Procedures: TB 700-2/NAVSEAINST 8020.8B/TO 11A-1-47/DLAR 8220.1-5 January (1998)].

In one aspect, this invention relates to a new process for producing bis-dinitroalkyl acetals and formals having the formula (1):

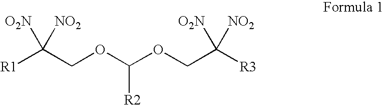

by oxidative nitration of compounds having the formula (2):

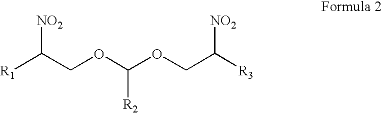

wherein R₁ and R₃ are independently C₁-C₄ straight or branched chain alkyl groups and R₂ is hydrogen, methyl, ethyl, n-propyl or isopropyl, preferably via a suitable alkali or alkaline earth metal nitronate salt of the compounds of Formula (2). The compounds of Formula (2) are all novel and form a further aspect of the invention. The nitronate salts also are new compounds. Compounds of Formula (1) in which R₁ and/or R₃ are C₃ or C₄ alkyl groups are novel and form another aspect of this invention. Also, compounds of Formula (1) in which R₂ is ethyl, n-propyl or isopropyl are novel and form yet another aspect of the invention. Additionally, compounds of Formula (1) in which R₁ and R₃ are both ethyl, or one of R₁ and R₃ is methyl and the other is ethyl, and R₂ is methyl are novel compounds and form still another aspect of the invention.

More specifically, an aspect of the invention relates to a process for producing BDNPA and/or BDNPF via oxidative nitration of the novel intermediates bis(2-nitro-1-propyl)acetal [BNPA] and/or bis(2-nitro-1-propyl)formal [BNPF], preferably via their sodium salts, or other suitable alkali metal or alkaline earth metal salts.

As indicated above, the process can be used to produce either BDNPA or BDNPF, but most preferably is used to produce a mixture of the two, forming A/F in one processing step by simultaneous oxidative nitration of their novel intermediates.

Another aspect of this invention is a process for producing compounds having the Formula (2) above, comprising reacting an aldehyde having the formula R₂CHO with a nitroalkanol having the formula R₁CH(NO₂)CH₂OH in the presence of a suitable catalyst.

### DETAILED DESCRIPTION OF THE INVENTION

In one aspect, this invention relates to a new process for producing bis-dinitroalkyl acetals having the formula (1):

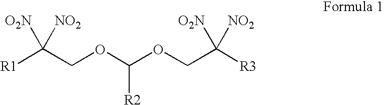

by oxidative nitration of compounds having the formula (2):

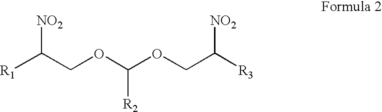

wherein R₁ and R₃ are independently C₁-C₄ straight or branched chain alkyl groups and R₂ is hydrogen, methyl, ethyl, n-propyl or isopropyl, preferably via a suitable alkali or alkaline earth metal nitronate salt of the compounds of Formula (2). The compounds of Formula (2) are all novel and form a further aspect of the invention. The nitronate salts also are new compounds. Compounds of Formula (1) in which R₁ and/or R₃ are C₃ or C₄ alkyl groups are novel and form another aspect of this invention. Also, compounds of Formula (1) in which R₂ is ethyl or propyl are novel and form yet another aspect of the invention. Additionally, compounds in which R₁ and R₃ are both ethyl, or one of R₁ and R₃ is methyl and the other is ethyl, and R₂ is methyl are novel compounds and form still another aspect of the invention.

As stated above, the invention also includes the novel intermediates having the Formula (2) and a process for producing them comprising reacting an aldehyde having the formula R₂CHO with a nitroalkanol having the formula R₁CH(NO₂)CH₂OH in the presence of a suitable catalyst.

More specifically, in the first-mentioned embodiment the invention relates to a process for producing BDNPA and/or BDNPF via oxidative nitration of the novel intermediates bis(2-nitropropyl-1)acetal [BNPA] and/or bis(2-nitropropyl-1)formal [BNPF], preferably via a suitable alkali or alkaline earth metal salt of such an intermediate, i.e. a nitronate salt. Preferred salts are the sodium, potassium and calcium nitronates of compounds of Formula (2).

In previously described processes, 2,2-Dinitropropanol [DNPOH] is the required intermediate to prepare A/F. DNPOH is regulated as a class 1.1 explosive. In the use of this invention the need to make DNPOH, or for that matter any explosive intermediate, is completely avoided, thus making the process safer. Because this invention eliminates the need to make explosive intermediates, quantity-distance restrictions regulating the design, construction and operation of the manufacturing plant do not apply, making this process less costly and available to any commercial chemical operation for its manufacture. Neither BNPA nor BNPF is an explosive.

In addition, in all previous processes for the synthesis of A/F it is necessary to prepare a geminal-dinitro moiety, DNPOH. Making the geminal-dinitro moiety is the most expensive part of the synthesis. In the process of this invention the geminal-dinitro moiety is put directly on the final product A/F. As a result, process losses of the most expensive material in the reaction sequence are significantly reduced. Because of this, the cost to prepare A/F will be significantly lower using the process of the invention.

Geminal-dinitro compounds of Formula (1) that may be made by the process and through the novel intermediates of this invention include the known compounds bis(2,2-dinitropropyl)acetal [BDNPA] (R₁, R₂, R₃=methyl), bis(2,2-dinitropropyl)formal [BDNPF] (R₁, R₃=methyl, R₂=hydrogen), -2,2-dinitrobutyl-2′,2′-dinitropropyl)formal [DNBPF] (R₁═CH₃; R₃═C₂H₅; R₂═H; see U.S. Pat. No. 4,997,499), and bis(2,2-dinitrobutyl)formal [BDNBF] (R₁, R₃═C₂H₅; R₂═H; see, e.g. U.S. Pat. No. 6,620,288). Compounds of Formula (1) in which R₁ and/or R₃ are C₃-C₄ alkyl are novel, as are the compounds 2,2-dinitrobutyl, 2′,2′-dinitropropylacetal [DNBPA] (R₁, R₂=methyl; R₃=ethyl) and bis(2,2-dinitrobutylacetal [BDNBA] (R₁, R₂=methyl; R₃=ethyl). These novel compounds will function in explosives and otherwise similarly to those compounds that are known. Compounds of Formula (2) are all novel.

In a preferred embodiment of this invention, the final product is A/F, which is generally composed of an eutectic solution ranging from about 45 to about 55 weight % BDNPA and about 45 to about 55 weight % BDNPF (most preferably about a 50/50 eutectic solution). Most preferably it is prepared in one reaction step, as a combined solution. In previously described processes, each A/F component, i.e., BDNPA and BDNPF, had to be prepared in separate processing steps using different condensation catalysts for each step and using different manufacturing equipment for its production. However, by the process of this invention A/F can be made simultaneously in one processing step using a single condensation catalyst from an approximately a 50/50 weight % solution of the new composition matter intermediates, BNPA and BNPF. The advantage of this facet of this invention is that the manufacturing cost for the A/F processing step is cut by nearly one-half. In addition, mixtures of BDNPA and BDNPF having ratios outside the 45-55% range may also be prepared simultaneously by this process.

One benefit of this invention can also be seen in the fact it is not necessary to isolate any of the intermediates in the pure or neat form. The intermediates can be made and used in the presence of a suitable solvent, such as alkyl ethers, e.g., di-isopropyl ether (IPE) or tertiary-butyl methyl ether (TBME), halogenated hydrocarbons, e.g., dichloromethane (MeCl₂) or 1,2-dichloroethane (EDC), hydrocarbons, e.g., octane or esters, e.g., ethyl acetate. However, if desired, the intermediates can be isolated.

The chemistry of the invention, depicted in the embodiment of a process for simultaneous production of A/F, and starting with 2-nitroethane, is shown in Scheme IV and described in the following paragraphs. However, production of A/F or other products according to this invention does not require starting with nitroethane, as nitroalcohols such as 2-nitro-1-propanol, 2-nitro-1-butanol and higher alcohols may be obtainable from commercial sources.

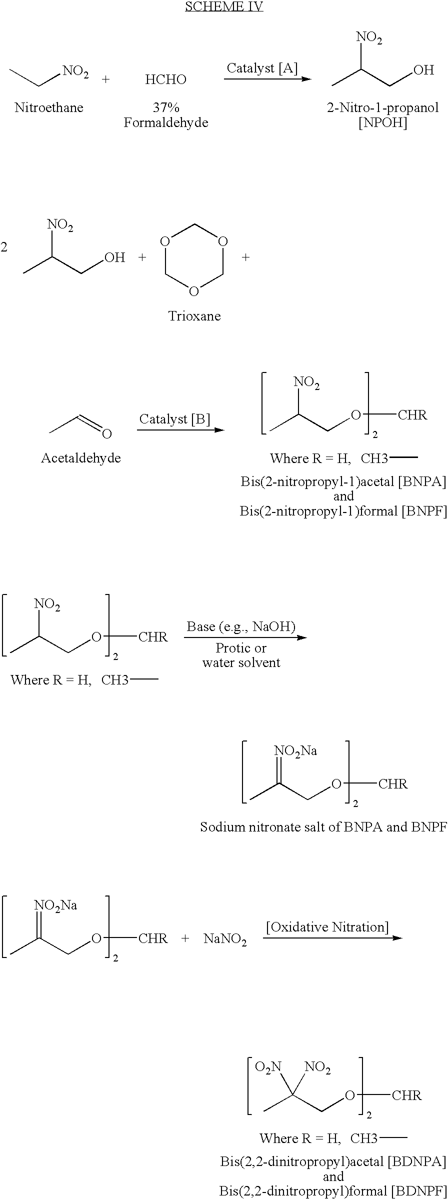

### Step I: Preparation of 2-Nitro-1-Propanol [NPOH]

The novel process of this invention begins with the reaction of a mononitroalkanol with formaldehyde or acetaldehyde, in one or another of their known forms. The mononitroalkanol may be purchased commercially or may be prepared, for example by the process shown in scheme IV represented by the production of 2-nitro-1-propanol from nitroethane.

As shown above in Scheme IV, 2-nitro-1-propanol may be prepared in a generally known process, by reaction of nitroethane with formaldehyde in a nitroaldol addition [i.e., a Henry Reaction (Henry, C.R. Acad. Sci. Paris, 120:1265 (1895)] in the presence of a catalyst. Typically the formaldehyde is used as an aqueous solution, e.g., a 37% aqueous solution of formaldehyde. The reaction temperature can range from ambient temperature to about 80° C. The preferred temperature is from about 45 to about 55° C. The mole ratio of nitroethane to formaldehyde can range from about 1:1 to about 2:1, respectively. The preferred molar ratio is about 2:1, i.e. a 100% molar excess of nitroethane. The preferred catalyst is sodium hydroxide or potassium hydroxide; however a number of other catalyst systems can be used.

The crude product, 2-nitro-1-propanol [NPOH] is obtained by extraction with a suitable solvent or by azeotropic distillation of the water present in the reaction. Solvents that can be used to conduct the azeotropic distillation include, but are not limited to, hexane, isopropyl ether, dichloromethane and 1,2-dichloroethane. The preferred solvent is hexane. The crude NPOH can be used in the next step of the process as is, or it may be first purified, for example by distillation at reduced pressure, preferably at 72-74° C. and 1 Torr.

Preparation of 2-nitro-1-butanol, a precursor for 2,2-dinitrobutyl compounds, can be carried out in a similar manner using 1-nitropropane as the starting material to prepare 2-nitro-1-butanol, or again this product can be purchased from commercial sources. Other alcohols from which novel compounds can be prepared may be prepared similarly or may be available from commercial sources.

### Step II: Conversion of NPOH into Bis(2-nitro-1-propyl)acetal (BNPA) and Bis(2-nitro-1-propyl)formal (BNPF)

BNPA and BNPF, which are novel compounds, can be made by the novel process generally shown in Scheme IV above and described in the following paragraphs.

BNPA is formed by the reaction of NPOH with either acetaldehyde or paraldehyde (the trimer form of acetaldehyde) in the presence of a condensation catalyst. The ratio of NPOH to aldehyde can range from about 3 to 1, respectively, to about 1 to 1 respectively; preferably from about 2 to 1 to 2.2 to 1, respectively. The preferred catalysts are (1) concentrated sulfuric acid, (2) a mixture of concentrated sulfuric acid and anhydrous magnesium sulfate, (3) boron trifluoride and (4) a boron trifluoride complex such as the diethyl etherate. When concentrated sulfuric acid is used as the catalyst, the molar ratio of NPOH to H₂SO₄ can range from about 1:3 to about 3:1, respectively; preferably from about 1:0.7 to about 1:0.1, respectively. When a mixture of concentrated sulfuric acid and anhydrous magnesium sulfate is the catalyst, the molar ratio of NPOH to H₂SO₄ to MgSO₄ can range from about 1:0.5:0.5, respectively to about 1:2:4, respectively, preferably from about 1:0.2:0.5 to about 1:0.1:0.1, respectively. When the catalyst is either boron trifluoride or a boron trifluoride complex such as the etherate, the molar ratio of NPOH to BF₃ can range from about 2:1 to about 1:4 respectively, preferably from about 1:0.8 to about 1:0.2, respectively. In this reaction, the catalyst is generally not recovered for recycle.

The temperature for the preparation of BNPA can range from about −20 to about 30° C., preferably from about −17 to about 0° C. The reaction time for the preparation of BNPA can range from about 5 minutes to about 4 hours, preferably from about 15 minutes to about 1 hour. The reaction can be conducted either with or without the presence of a solvent; however, it is preferred that a solvent be used. The solvent may be chosen from aliphatic hydrocarbons, alkyl ethers (either symmetrical or unsymmetrical) and halogenated hydrocarbons. The preferred solvents are isopropyl ether, methyl-t-butyl ether, hexane, dichloromethane and 1,2-dichloroethane.

BNPF is similarly prepared from NPOH under the same conditions and using the same type of catalysts, but using formaldehyde (preferably as the trimer, trioxane) or its polymeric form, paraformaldehyde. Catalysts, reaction times, and other conditions are as described above for the preparation of BNPA, except that the temperature would be from about −20 to about 70° C., preferably from about −17 to about 30° C.

BNPA and BNPF can be synthesized either independently or simultaneously. In a preferred embodiment of the invention, BNPA and BNPF are prepared together at the same time, in the same process steps, by adding both formaldehyde and acetaldehyde to NPOH. The formaldehyde may be in the form of the trimer (trioxane) or the polymer (paraformaldehyde); the acetaldehyde may be independently in the form of acetaldehyde (monomer) or paraldehyde (trimer). The mole ratio of NPOH to acetaldehyde to formaldehyde can range from about 3:1:1, respectively, to about 1:1:1 respectively; preferably from about 2:1:1 to about 2:0.3:0.3, respectively. Preferably the mole ratio of formaldehyde to acetaldehyde is about 1:0:1.0. The preferred catalysts are (1) concentrated sulfuric acid, (2) a mixture of concentrated sulfuric acid and anhydrous magnesium sulfate, (3) boron trifluoride and (4) boron trifluoride complexes such as the diethyl etherate. When concentrated sulfuric acid is used as the catalyst, the molar ratio of NPOH to H₂SO₄ can range from about 1:3 to about 3:1, respectively, preferably from about 1:0.7 to about 1:0.1, respectively. When a mixture of concentrated sulfuric acid and anhydrous magnesium sulfate is the catalyst, the molar ratio of NPOH to H₂SO₄ to MgSO₄ can range from about 1:0.5:0.5, respectively to about 1:2:4, respectively, preferably from about 1:0.2:0.5 to about 1:0.1:0.1, respectively. When the catalyst is either boron trifluoride or boron trifluoride etherate, the molar ratio of NPOH to BF₃ can range from about 2:1 to about 1:4 respectively, preferably from about 1:0.8 to about 1:0.2, respectively. In this reaction, the catalyst is generally not recovered for recycle.

The temperature for the simultaneous preparation of BNPA/BNPF can range from about −20 to about 30° C., preferably from about −17 to about 0° C. The reaction time can range from about 5 minutes to about 4 hours, preferably from about 15 minutes to about 1 hour. This will result in a product containing the combined BNPA/BNPF to be “tailor-made” to any desired composition of each component. This is accomplished by varying the mole ratio of acetaldehyde to formaldehyde. On completion of the reaction, the catalyst is removed by any suitable number technique such as decantation, phase separation and filtration. The spent catalyst is discarded. The resulting supernatant liquid is washed first with an alkaline base in water, preferably sodium bicarbonate, then with water and then the solvent is removed by distillation, preferably under reduced temperature and pressure, from about 45 to about 55° C. and about 1 Torr. The resulting product, containing the crude BNPA and BNPF, can be used directly in the subsequent reaction.

Similarly, nitrobutyl and other compounds of Formula (II) within the scope of this invention can be made by using the appropriate starting materials produced from 2-nitro-1-butanol or homologous alcohols.

To form mixed acetals and formals two different starting alcohols are required. Previous work [Cho, et. al., U.S. Pat. No. 6,620,268; Sep. 16, 2003] and the references therein have shown that mixed formals result when 2,2-dinitropropanol and 2,2-dinitro-1-butanol are co-reacted with formaldehyde (trioxane) in the presence of a condensation catalyst. Mixed acetals can also be formed by the same mechanism starting with acetaldehyde. The present invention will also afford mixed formals and acetals of the corresponding mononitro alcohols by the same technique. For example, by reacting a mixture of 2-nitro-1-propanol and 2-nitro-1-butanol with either formaldehyde (trioxane) or acetaldehyde the mixed acetals and formals of formula (2) in which R₁ is methyl and R₃ is ethyl will be formed. Furthermore, if the reaction of the mixed mononitro alcohols is done in the presence of acetaldehyde and formaldehyde together the resulting product will be the mixed acetals and formals of those two mononitro alcohols. The starting alcohols are well known and have been reported in literature. [for example, J. Org. Chem., 8(1), 7 (1943); JACS, 71(8), 2947 (1949); ibid, 72(7), 3241 (1950); ibid, 68(9), 1519 (1945)]. Other mixed acetals and formals can be similarly prepared from the appropriate mononitro alcohols.

### Step III: Conversion of BNPA/BNPF into A/F

A BNPA/BNPF mixture or the individual components is converted into A/F (or A or F, respectively) by an oxidative nitration process. In this process BNPA and BNPF are converted into the corresponding nitronate salts by treatment of the components either with an aqueous sodium hydroxide solution in the presence of methanol or with a sodium alkoxide, such as sodium methoxide, in the presence of methanol or ethanol. The nitronate salt of each component is converted into the corresponding geminal-dinitro compound, i.e. BDNPA and BDNPF, on treatment with either a solution of silver nitrate and sodium nitrate or a solution of potassium ferricyanide and sodium nitrate. The preferred method is to use silver nitrate with sodium nitrate. The formation of the nitronate salt and its conversion to the geminal dinitro compound are conducted in sequential reactions.

The temperature of the reaction can range from about −10 to about 80° C., preferably from about 5 to about 30° C. The reaction time can range from about 5 minutes to about 5 hours, preferably from about 15 minutes to about 2 hours. The equivalent ratio (respectively) of BNPA or BNPF or a mixture of BNPA/BNPF to sodium hydroxide to silver nitrate to sodium nitrite can range from about 1.0:1.0:2:0:1.0 to about 1.0:1.5:2.2:2.0, and preferably is about 1.0:1.01:2.01:1.2. The amount of water or protic solvent in the reaction can range from about 25% by weight to about 90% by weight; preferably from about 40 to about 70% by weight. A solvent may be present during the reaction. If a solvent is used, it is preferably added after the reaction is completed. The solvent is chosen from aliphatic hydrocarbons, alkyl ethers (either symmetrical or unsymmetrical) and halogenated hydrocarbons. The preferred solvents are isopropyl ether, methyl-t-butyl ether, hexane, dichloromethane and 1,2-dichloroethane. The final product (A/F) solvent solution may be isolated by decantation, phase separation or filtration and the supernatant liquid containing the A/F is washed with an aqueous alkaline solution. The preferred alkaline solution is a 3 to 10% aqueous solution of sodium hydroxide. The solvent is removed under reduced pressure, at 45 to 75° C. and 1 Torr to give crude product, A/F. The crude A/F can be used without further purification. If desired, the crude A/F can be purified by distillation, preferably by short-path distillation using a “wiped-film-still” at about 100-110° C. and about 20-50 microns (Hg) pressure.

Similarly, other compounds of the type defined by Formula (1) can be made using the appropriate starting materials.

### EXAMPLES

The following examples illustrate the invention but are not intended to serve as limitations on the principles of the invention. This description will clearly enable one skilled in the art to make and use the invention, and describes several embodiments, adaptations, variations, alternatives and uses of the invention, including what is presently believed to be the best mode of carrying out the invention.

The reactants are identified throughout by chemical nomenclature with reference to typical commercially available sources by way of example and not by way of limitation. The reactants are also shown in structural format in the chemical equations and a description of the process is shown in the Process Flow Diagram. It will be recognized by one skilled in the art that the reaction products may be single molecular species, or more complex mixtures of possible reaction products. Thus, while we have shown in the chemical equations the structural formulas of reaction products, those are by way of example and not by way of limitation of the actual or possible products of the process using the reactants shown in the equations. Accordingly, the invention, without limitation, covers novel and/or new composition of matter components to produce A/F.

### Preparation of BIS(2-NITRO-1-PROPYL)FORMAL (BNPF)

### Experiment 1

### Octane Solvent and p-Toluenesulfonic Acid Condensation Catalyst

Into a 500-ml 3-neck reaction flask, equipped with mechanical stirrer, thermometer, electrical heating mantle and a condenser-Dean-Stark Trap were put 150 ml of n-octane, 1.80 g (0.06 equivalents as formaldehyde), 12.62 g (0.12 mol) of 2-nitro-1-propanol (NPOH) (Aldrich 97%) and 0.3 grams of p-toluenesulfonic acid. Two phases were formed in the reaction mixture. The reaction mixture was brought to reflux to azeotropically distill the water formed during the reaction. Water was removed over a period of 30 minutes, during which time the pot temperature rose from 89 to 124° C. and 1.05 ml of water was collected. Based on the water recovered, the conversion of the 2-nitro-1-propanol was nearly quantitative. The reaction mixture was allowed to cool to 26° C. over a 30-minute period and mixing was stopped to allow the two layers to separate. The heavier bottom layer containing the product, BNPF, was removed (11.96 g, 90% crude yield), dissolved in 45 ml of dichloromethane (MeCl₂) and washed with 20 ml of 7% sodium bicarbonate and 70 ml of distilled water to a pH of 6. The BNPF/MeCl₂ solution was dried over anhydrous sodium sulfate, filtered (cake washed with more MeCl₂) and concentrated in vacuo to give 7.63 g (57.3% crude yield) of BNPF, which assayed 91.0% (area %) by glc analysis and had an N_{D} (Jiang, et. al., Tetrahedron Lett., 45:2699-2701 (2004)) of 1.4510. The 33% drop in yield was attributed to the solubility of the BNPF in water.

### Experiment 2

### Dichloromethane Solvent and Concentrated Sulfuric Acid Catalyst

Into a 500-ml 3-neck reaction flask, equipped with mechanical stirrer, thermometer, pressure equalizing addition funnel and ice-water bath were put 150 ml of MeCl₂, 1.80 g (0.06 equivalents as formaldehyde) and 12.61 g (0.12 mol) of 2-nitro-1-propanol (NPOH) (Aldrich 97%) and mixed to achieve solution. The solution was cooled to 15° C. in an ice-water bath. With vigorous stirring, 15 g (0.15 mol) of sulfuric acid (98%) was added dropwise over a 14-minute period, during which time the temperature rose to 20° C. The ice bath was removed and the reaction mixture was stirred for an additional 1.5 hours, while letting the temperature climb to 28° C. (room temperature). Mixing was stopped to allow the two layers to separate. The heavier bottom layer containing the sulfuric acid was removed (19.72 g) and the MeCl₂ layer, containing the product BNPF, was washed with two 100-ml portions of 7% sodium bicarbonate. The aqueous portions of the extract were combined and extracted with one 50-ml portion of MeCl₂. The MeCl₂ portions were combined, dried over sodium sulfate (cake washed with more MeCl₂), filtered and concentrated in vacuo (35-40° C. at 1.5 torr) to give 8.83 g (66.3% crude yield) of BNPF (straw colored liquid) which assayed 83.4% (area %) by glc analysis and had an N_{D} (Jiang, et. al., Tetrahedron Lett., 45:2699-2701 (2004))25 of 1.4485.

### Preparation of BIS(2,2-DINITROPROPYL)FORMAL (BDNPF)

### Experiment 3

### Potassium Ferricyanide Oxidative Nitration Method

Into a 500-ml reaction flask (described above ) were added 16.93 g (0.0521 mol) of potassium ferricyanide and 7.19 g (0.104 mol) sodium nitrite all dissolved in 100 ml of distilled water. To this solution was added 50 ml of MeCl₂ and the two-phase mixture was cooled to 5° C. To this mixture, with vigorous agitation were then added in one portion a solution containing 1.11 g (0.005 mol) BNPF (made as described above), 20 ml of water, 25 ml of methanol and 0.45 g (0.0113 mol) of sodium hydroxide. The contact (stirring) time of the BNPF with the sodium hydroxide solution was only 15 seconds; then it was added to the potassium ferricyanide/sodium nitrite solution. The reaction temperature was kept at 5° C. with intermittent cooling with a dry ice/methanol cooling bath. The cooling was then removed and the reaction mixture allowed to climb to 12° C. over a 25 minute period, then allowed to come to room temperature (25° C.). The bottom organic layer was removed and the top aqueous layer was extracted with one 50-ml portion of MeCl₂. The organic layer and the MeCl₂ extract were combined and washed with two 50-ml portions of water. The last water wash had a pH of about 7. The MeCl₂-BDNPF solution was dried over sodium sulfate, as described earlier, then concentrated in vacuo (40° C. at 2 torr) to give 1.06 g (67.9% yield) of crude BDNPF, which was shown to contain only 20.5% BDNPA and 65.3% starting material (BNPF) by glc area %. It should be noted that, as described below, the amount of time the starting material BNPF is allowed to be in contact with the base (NaOH) can have a significant effect on the yield of BDNPF or BDNPA.

### Preparation of BIS(2-NITRO-1-PROPYL)ACETAL (BNPA)

### Experiment 4

### Dichloromethane Solvent and Boron Trifluoride Etherate Catalyst

In a similar set-up to that described in Experiment 2, except that dry ice/methanol was used in place of the wet ice bath, were added 150 ml of MeCl₂, 12.61 g (0.12 mol) of 2-nitro-1-propanol and 2.96 g (0.067 mol) of freshly distilled acetaldehyde. The solution was cooled to −17° C. and 10.22 g (0.072 mol) of BF₃.Et₂O was added dropwise over a 12-minute period, keeping the reaction temperature between −17 and -14° C. The reaction solution was stirred for an additional 22 minutes, allowing the temperature to rise to −8° C. The reaction solution was then quenched with 75 ml of cold distilled water, stirred for 6 minutes and the temperature was allowed to climb to 8° C. The layers were separated. The top aqueous layer had a pH of 1.5. The bottom organic layer was washed with three 100-ml portions of 7% sodium bicarbonate while allowing the reaction solution to come to room temperature. The pH of the last wash was 8 to 9 (as determined by pH paper}. The organic layer was dried over sodium sulfate as before and then concentrated in vacuo at 39° C. at 2 torr to give 9.2 g (64.9% crude yield) of BNPA which assayed 89.9% (area %) by glc analysis and had an N_{D} (Henry, C.R. Acad. Sci. Paris, 120:1265 (1895)) of 1.4481.

### Preparation of BIS(2,2-DINITROPROPYL)ACETAL (BDNPA)

### Experiment 5

### Potassium Ferricyanide Oxidative Nitration Method

Into a 500-ml reaction flask (described above) were added 16.93 g (0.0521 mol) of potassium ferricyanide and 7.19 g (0.104 mol) sodium nitrite all dissolved in 100 ml of distilled water. To this solution were added 50 ml of MeCl₂ and the two-phase mixture was cooled to 5° C. To this mixture, with vigorous agitation, were then added in one portion a solution of 1.10 g (0.0047 mol) BNPA (Reference 205-A/F-8) dissolved in solution of 15 ml of water, 10 ml of methanol and 0.45 g (0.0113 mol) of sodium hydroxide. The reaction temperature rose to 7° C. The reaction mixture was stirred for 25 minutes at 10 to 12° C., then allowed to come to room temperature (25° C.). The bottom organic layer was removed and the top aqueous layer was extracted with one 50-ml portion of MeCl₂. The MeCl₂ portions were combined and washed with two 50-ml portions of water. The last water wash had a pH of about 7 (paper). The MeCl₂-BDNPA solution was dried over sodium sulfate, as described earlier, then concentrated in vacuo (40° C. at 0.8 torr) to give 1.07 g (68.6% yield) of crude BDNPA which analyzed only 13.6% BDNPA and 81.7% starting material (BNPA) by glc area %. As mentioned above, the amount of time the starting material, BNPA is allowed to be in contact with the base, NaOH, is shown to have a significant effect on the yield of BDNPA or BDNPF.

### Simultaneous Preparation of BNPA and BNPF

### Experiment 6

### Dichloromethane Solvent and Concentrated Sulfuric Acid Catalyst

Into a 500-ml 3-neck reaction flask, equipped with mechanical stirrer, thermometer, pressure equalizing addition funnel and dry-ice-methanol cooling bath, was put 150 ml of MeCl₂ (room temperature). Then 1.09 g (0.04 equivalents as formaldehyde) of trioxane and 12.61 g (0.12 mol) of 2-nitro-1-propanol (Aldrich 97%) were added and mixed at room temperature to achieve solution. The solution was cooled to 1° C. with the dry-ice-methanol bath and 1.59 g (0.036 mol) of freshly distilled acetaldehyde was added; then the solution was cooled to −17° C. With vigorous stirring, 15 g (0.15 mol) of sulfuric acid (98%) was added dropwise over a 12-minute period, keeping the temperature between −13 and -17° C. The reaction mixture was stirred for an additional 78 minutes at −13 to −17° C. The sulfuric acid layer became a pasty sludge during this time. The organic layer (about 150 ml) was allowed to warm to −9° C., then was decanted from the sulfuric acid sludge (20.1 g), transferred into a separatory funnel and quenched with 100 ml of cold (5° C.) sodium bicarbonate solution (7%). No foaming was observed during the quench procedure. The organic layer was washed with two more 100-ml portions of 7% sodium bicarbonate, allowing the temperature to come to ambient conditions (about 23° C.). The organic layer was dried over sodium sulfate (cake washed with more MeCl₂), filtered and concentrated in vacuo (35-40° C. at 1.0 torr) to give 9.11 g (66.0% crude yield) of a solution with an approximate BNPA/BNPF weight ratio of 50/50. The concentration of the BNPA/BNPF in the concentrate was found to be 82.3% (area %) by glc analysis and had an N_{D} (Jiang, et. al., Tetrahedron Lett., 45:2699-2701 (2004)) of 1.4504.

### Experiment 7

### Dichloromethane Solvent and Boron Trifluoride-Etherate Catalyst

Into 500-ml 3-neck reaction flask, equipped with mechanical stirrer, thermometer, pressure equalizing addition funnel and dry-ice-methanol cooling bath, was put 150 ml of MeCl₂ (room temperature). Then 1.09 g (0.04 equivalents as formaldehyde) of trioxane and 12.61 g (0.12 mol) of 2-nitro-1-propanol (Aldrich 97%) were added and mixed at room temperature to achieve solution. The solution was cooled to 1° C. with the dry-ice-methanol bath and 1.59 g (0.036 mol) of freshly distilled acetaldehyde was added; then the solution was cooled to −17° C. With vigorous stirring, 10.22 g (0.07 mol) of sulfuric BF₃-etherate was added dropwise over a 12 minute period, keeping the temperature at about −15° C. On completion of the addition, the reaction solution was stirred for an additional 18 minutes at about −14° C., then was quenched with 75 ml of distilled water, allowing the temperature to climb to 5° C. The quenched reaction mixture was transferred into a separatory funnel; the phases were separated and the organic layer was washed with three 100-ml portions of 7% sodium bicarbonate, allowing the temperature to come to ambient conditions (about 23° C.). The organic layer was dried over sodium sulfate (cake washed with more MeCl₂), filtered and concentrated in vacuo (39° C. at 1.1 torr) to give 8.3 g (60.0% crude yield) of a dark brown solution with an approximate BNPA/BNPF weight ratio of 50/50. The concentrate was found to contain 83.4% BNPA/BNPF and 10.5% unreacted 2-nitro-1-propanol by glc analysis (area %) and had an N_{D} ( )25 of 1.4484.

### Experiment 8

### Dichloromethane Solvent with Reduced Amount of Concentrated Sulfuric Acid Catalyst and Anhydrous Magnesium Sulfate as the Condensation Catalyst

Into a 500-ml 3-neck reaction flask, equipped with mechanical stirrer, thermometer, pressure equalizing addition funnel and dry-ice-methanol cooling bath, was put 120 ml of MeCl₂ (room temperature). Then 0.78 g (0.026 equivalents as formaldehyde) of trioxane and 9.00 g (0.086 mol) of 2-nitro-1-propanol (Aldrich 97%) were added and mixed at room temperature to achieve solution. The solution was cooled to −2° C. with the dry-ice-methanol bath and 10.71 g of anhydrous magnesium sulfate and 1.13 g (0.026 mol) of freshly distilled acetaldehyde was added; then the slurry was cooled to about −18° C. With vigorous stirring, 3.57 g (0.037 mol) of sulfuric acid (98%) was added dropwise over a 2-minute period, during which time the temperature rose to −16° C. The reaction mixture was stirred for an additional 120 minutes at −11 to −17° C. It was noticed that the color of the reaction slurry was lighter than in other similar reactions and no slush or freezing was formed. The organic layer was decanted from the sulfuric acid/magnesium sulfate slurry and transferred, with stirring, into 100 ml of cold (5° C.) sodium bicarbonate solution (7%). No foaming was observed during the quench procedure. The organic layer was transferred into a separatory funnel and washed with two more 100-ml portions of 7% sodium bicarbonate, allowing the temperature to come to ambient conditions (about 23° C.). The organic layer was dried over sodium sulfate (cake washed with more MeCl₂), filtered and concentrated in vacuo (40° C. at 1.6 torr) to give 7.60 g (77.4% crude yield) of a solution with an approximate BNPA/BNPF weight ratio of 50/50. The concentration of the BNPA/BNPF in the concentrate was found to be 95.1% (area %) by glc analysis and had an N_{D} (Jiang, et. al., Tetrahedron Lett., 45:2699-2701 (2004))₂₅ of 1.4478. All but 1% of the starting 2-nitro-1-propanol was converted, as determined by glc (area %) analysis.

### Simultaneous Preparation of BDNPA and BDNPF

### Experiment 9

### Potassium Ferricyanide Oxidative Nitration Method in the Presence of Dichloromethane

Into a 500-ml reaction flask (described above ) were added 16.93 g (0.0521 mol) of potassium ferricyanide and 7.19 g (0.104 mol) sodium nitrite, all dissolved in 100 ml of distilled water. To this solution were added 50 ml of MeCl₂ and the two-phase mixture was cooled to 4° C. To this mixture, with vigorous agitation were then added in one portion a solution of 0.55 g (0.00224 mol) BNPA and 0.55 g (0.00248 mol) of BNPF (both prepared as described above), both dissolved in a solution of 25 ml of water, 20 ml of methanol and 0.45 g (0.0113 mol) of sodium hydroxide. The contact (stirring) time of the BNPA and BNPF with the sodium hydroxide solution was for 15 minutes before it was added to the potassium ferricyanide/sodium nitrite solution. The reaction temperature was kept at 4° C. with intermittent cooling with a dry ice/methanol cooling bath; then the cooling was removed and the reaction mixture allowed to climb to 11.5° C. over a 25-minute period, then allowed to come to room temperature (25° C.). The bottom organic layer was removed and the top aqueous layer was extracted with 50 ml MeCl₂. The organic layer and the MeCl₂ extract was combined and washed with two 50-ml portions of water. The last water wash had a pH of about 7 (paper). The MeCl₂-BDNPF solution was dried over sodium sulfate, as described earlier, then concentrated in vacuo (40° C. at 1.4 torr) to give 1.16 g (74.4% yield) of crude BDNPA/BDNPF which analyzed 25.5% BDNPA/BDNPF and 54.5% starting material (BNPA/BNPF) by glc area %. It was apparent that the extra time (i.e., 15 minutes vs. 15 seconds) the starting material, BNPA/BNPF was allowed to be in contact with the base, NaOH, increased the yield of BDNPF/BDNPA.

### Experiment 10

### Potassium Ferricyanide Oxidative Nitration Method without the Presence of Dichloromethane during the Reaction

Into a 500-ml reaction flask (described in Experiment 5) were added 16.93 g (0.0521 mol) of potassium ferricyanide and 7.19 g (0.104 mol) sodium nitrite all dissolved in 100 ml of distilled water and cooled to 4° C. To this solution, with vigorous agitation were then added in one portion a solution of 1.10 g (0.0048 mol) of the 50/50 mixture of BNPA/BNPF that was simultaneously made in Experiment 6 (Reference 205-A/F-15) dissolved in solution of 25 ml of distilled water, 18 ml of methanol and 0.45 g (0.0113 mol) of sodium hydroxide at 5° C. The contact (stirring) time of the BNPA and BNPF with the sodium hydroxide solution was 30 minutes at 5° C. before it was added to the potassium ferricyanide/sodium nitrite solution. The reaction mixture was stirred for an additional 15 minutes at 4° C. MeCl₂ (50 ml) was added to the reaction mixture and then stirred for an additional 30 minutes, allowing the temperature to climb to 19° C. The bottom organic layer was removed and the top aqueous layer was extracted with 50 ml MeCl₂. The organic layers were combined and washed with two 50-ml portions of water. The MeCl₂-BDNPA/BDNPF solution was dried over sodium sulfate, as described earlier, then concentrated in vacuo (40° C. at 1.2 torr) to give 1.05 g (68% yield) of crude BDNPA/BDNPF which analyzed 76% BDNPA/BDNPF by glc (area %) and had an N_{D} (Jiang, et. al., Tetrahedron Lett., 45:2699-2701 (2004))₂₅ of 1.4660.

### Experiment 11

### Potassium Ferricyanide Oxidative Nitration Method with Extended Reaction Time

Into a 500-ml reaction flask (described in Experiment 10) were added 16.93 g (0.0521 mol) of potassium ferricyanide and 7.19 g (0.104 mol) sodium nitrite all dissolved in 100 ml of distilled water and cooled to 4° C. To this solution, with vigorous agitation were then added in one portion a solution of 1.10 g (0.0048 mol) of the 50/50 mixture of BNPA/BNPF that was simultaneously made in Experiment 6 dissolved in a solution of 25 ml of Distilled water, 18 ml of methanol and 0.45 g (0.0113 mol) of sodium hydroxide at 5° C. The contact (stirring) time of the BNPA and BNPF with the sodium hydroxide solution was for 30 minutes at 5° C. before it was added to the potassium ferricyanide/sodium nitrite solution. The reaction mixture was stirred for an additional 15 minutes at 4° C., then stirred for an additional 75 minutes, allowing the temperature to climb to 21° C. MeCl₂ (50 ml) was added to the reaction mixture and then stirred for an additional 3 minutes. The bottom organic layer was removed and the top aqueous layer was extracted with 50 ml MeCl₂. The organic layers were combined and washed with two 50-ml portions of water. The MeCl₂-BDNPA/BDNPF solution was dried over sodium sulfate, as described earlier, then concentrated in vacuo (40° C. at 1.4 torr) to give 1.40 g (91% yield) of crude BDNPA/BDNPF which analyzed 84% BDNPA/BDNPF by glc (area %) and had an N_{D} (Jiang, et. al., Tetrahedron Lett., 45:2699-2701 (2004))25 of 1.4663. This experiment demonstrates the importance of extended post-addition reaction time.

### Experiment 12

### Silver Nitrate Oxidative Nitration Method with Extended Reaction Time and Isopropyl Ether Extraction

Into a 200-ml beaker was added 6.54 g (0.0385 mol) of silver nitrate dissolved in 12 ml of distilled water and cooled to 5° C. In a separate container were dissolved 0.78 g (0.0195 mol) NaOH, 2.67 g (0.0.0387 mol) sodium nitrite and 10 ml distilled water and cooled to 5° C. To the sodium hydroxide/sodium nitrite solution were added 2.2 g (0.0096 mol) of the 50/50 mixture of BNPA/BNPF made in Experiment 7 and stirred for 30 minute; then 10 g of methanol were added and stirred for 45 minutes at about 6° C. to achieve total solution. The cold silver nitrate solution was stirred with a magnetic stirrer and the BNPA/BNPF solution was added over a period of about 45 seconds, during which time the temperature rose to 16° C. The reaction slurry was stirred for an additional 130 minutes, allowing the temperature to climb to 24° C. The reaction slurry was extracted first with 50 ml, then 35 ml, of isopropyl ether. The combined ether extracts were washed with 35 ml of water then dried over sodium sulfate, as described earlier, then concentrated in vacuo (40° C. at 0.8 torr) to give 2.36 g (77% yield) of crude BDNPA/BDNPF which analyzed 95.1% BDNPA/BDNPF by glc (area %) and had an N_{D} (Jiang, et. al., Tetrahedron Lett., 45:2699-2701 (2004)) of 1.4610.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound having the formula (2): 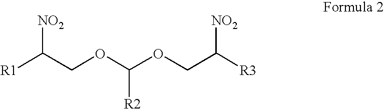
in which R₁ and R₃ are independently C₁-C₄ straight or branched chain alkyl and R₂ is hydrogen, methyl, ethyl, n-propyl or isopropyl.

2. A compound according to claim 1 in which R₂ is hydrogen.

3. A compound according to claim 1 in which R₂ is methyl.

4. A compound according to claim 1 in which R₁ and R₃ are both methyl.

5. A compound according to claim 4 in which R₂ is hydrogen.

6. A compound according to claim 4 in which R₄ is methyl.

7. A composition of matter comprising a mixture of compounds according to claims 5 and 6.

8. An alkali metal or alkaline earth metal salt of a compound according to claim 1.

9. A sodium salt of a compound according to claim 1.

10. A potassium salt of a compound according to claim 1.

11. A compound having the formula (1): 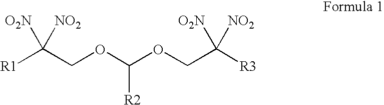
in which R₁ and R₃ are independently straight or branched chain C₃-C₄ alkyl groups; and R₂ is hydrogen, methyl, ethyl, n-propyl or isopropyl.

12. A compound having the formula (1): 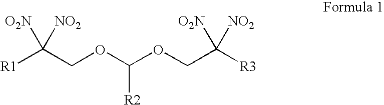
in which R_{1 l and R}₃ are both ethyl, or in which R₁ is methyl and R₃ is ethyl, and R₂ is methyl.

13. A compound having the formula (1): Page 3 of 11 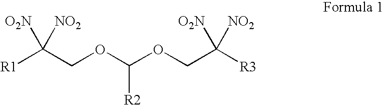
in which R₁ and R₃ are both ethyl, or in which R₁ is methyl and R₃ is ethyl, and R₂ is ethyl, n-propyl or isopropyl.

14. A process for simultaneously producing a compound of the formula 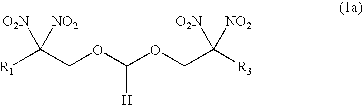
and a compound of the formula 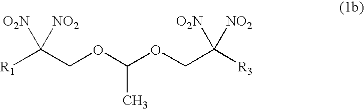
said process comprising simultaneously, and in a single solution, oxidatively nitrating a compound of the formula 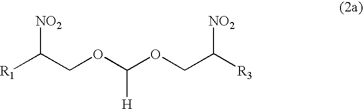
and a compound of the formula 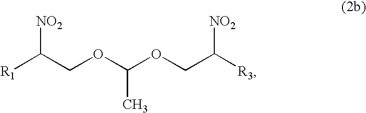
or alkali metal or alkaline earth metal salts of the compounds of Formulas (2a) and (2b),
in which R₁ and R₃ are independently straight or branched chain C₁-C₄ alkyl groups.

15. A process according to claim 14 in which the oxidative nitration is conducted in the presence of silver nitrate and sodium nitrite.

16. A process according to claim 14 in which the oxidative nitration is conducted in the presence of sodium nitrite and potassium ferricyanide.

17. A process according to claim 16 further comprising conducting the oxidative nitration in the presence of sodium persulfate.

18. A process according to claim 14 in which the compounds of Formulas (2a) and (2b) are first contacted with sodium hydroxide in an aqueous system, and then the resulting products are contacted with potassium ferricyanide and sodium nitrite.

19. A process according to claim 14 in which the compounds of Formulas (2a) and (2b) are first contacted with sodium hydroxide or a sodium alkoxide in the presence of water or a protic solvent to produce a nitronate salts of said compounds, and the nitronate salts are contacted with potassium ferricyanide and sodium nitrite.

20. A process according to claim 14 in which the compounds of Formulas (2a) and (2b) are first contacted with potassium hydroxide or a potassium alkoxide in the presence of water or a protic solvent to produce a nitronate salts of said compounds, and the nitronate salts are contacted with potassium ferricyanide and sodium nitrite.

21. (canceled)

22. (canceled)

23. (canceled)

24. (canceled)

25. (canceled)

26. A process according to claim 14 in which said process produces a mixture of from about 45 to about 55 weight % of a compound of Formula (1a) and from about 55 to about 45 weight % of a compound of Formula (1b).

27. A process for the production of compounds having the formula (2): 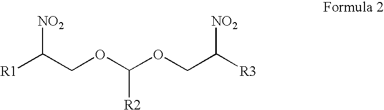
in which R₁ and R₃ are independently straight or branched chain C₁-C₄ alkyl groups and R₂ is hydrogen, methyl, ethyl, n-propyl or isopropyl,
comprising reacting an aldehyde having the formula R₂CHO with a nitroalkanol having the formula R₁CH(N0₂)CH₂OH, in the presence of a suitable catalyst.

28. A process according to claim 27 in which the nitroalkanol is 2-nitropropanol.

29. A process according to claim 27 in which the aldehyde is formaldehyde.

30. A process according to claim 29 in which the formaldehyde is in the form of trioxane.

31. A process according to claim 29 in which the formaldehyde is in the form of an aqueous solution of formaldehyde.

32. A process according to claim 29 in which the formaldehyde is in the form of paraformaldehyde.

33. A process according to claim 27 in which the aldehyde is acetaldehyde.

34. A process according to claim 33 in which the acetaldehyde is in the form of paraldehyde.

35. A process according to claim 27 in which the nitroalkanol is 2-nitrobutanol.

36. A process according to claim 27 in which the catalyst comprises concentrated sulfuric acid or a mixture of concentrated sulfuric acid and magnesium sulfate.

37. A process according to claim 27 in which the catalyst comprises boron trifluoride.

38. A process according to claim 27 in which the catalyst comprises a boron trifluoride complex.
